(19) United States
(12) Patent Application Publication
(10) Pub. No.: US 2011/0059925 A1
(43) Pub. Date: Mar. 10, 2011

(54) METHODS FOR IMPROVING LID MARGIN AND TEAR FILM FUNCTION AND TREATMENT OF LID MARGIN DISEASE USING TETRACYCLINE FAMILY ANTIBIOTICS

(76) Inventor: Eric Donnenfeld, Roslyn, NY (US)

(21) Appl. No.: 12/934,308

(22) PCT Filed: Mar. 26, 2009
(86) PCT No.: PCT/US09/38428

§ 371 Date: Nov. 23, 2010

Related U.S. Application Data
(60) Provisional application No. 61/070,850, filed on Mar. 26, 2008.

(51) Int. Cl. A61K 31/65

(52) U.S. Cl. 514/154; 514/152

(57) ABSTRACT

The present invention provides a mucoadhesive broad spectrum antibiotic with anti-inflammatory characteristics with strong tissue penetration for improving lid margin function and the treatment of diseases associated therewith. The present invention further provides compositions and methods for treating and/or preventing the signs and/or symptoms of blepharitis and dry eye disease.

### REFERENCE TO RELATED APPLICATIONS

This non-provisional patent application claims priority to U.S. Provisional Patent Application Ser. No. 61/070,850, filed Mar. 26, 2008, which is incorporated by reference herein in its entirety.

### FIELD OF THE INVENTION

The present invention relates generally to novel compositions and methods for the treatment and prevention of lid margin disease. More specifically, the present invention relates a composition comprising an anti-infective/anti-inflammatory agent and a mucoadhesive vehicle for improving lid margin function, thereby treating lid margin and tear film disease and diseases associated therewith, and methods of administering said compositions to a subject in need thereof.

### BACKGROUND

The lid margin plays a vital role in the production of tear film and maintenance of a healthy ocular surface. Lipids which are secreted by the meibomian glands in the eyelids contribute to a mixture along with mucin (secreted by the goblet cells in the conjunctiva, and an aqueous component (secreted by the lacrimal glands) which protects the cornea and serves as the major refracting surface of the eye.

Lid margin disease is a common, chronic disease with a broad and potentially serious impact. The signs and symptoms associated with lid margin disease can affect quality of life, and its sequelae can result in permanent tissue damage and loss of vision. Lid margin disease is also an important risk factor for complications and suboptimal outcomes in patients undergoing ocular surgery. Lid margin disease is also a common cause, or co-variant in another common ocular surface disease condition, dry eye. Lid margin disease can be associated with dry eye and/or can be a cause of dry eye, specifically, evaporative dry eye.

Lid margin disease is underdiagnosed and commonly misdiagnosed. Little is known about the underlying pathophysiology of lid margin disease and it is difficult to characterize as it frequently co-exists with other ocular conditions. In addition, therapeutic efficacy has been difficult to achieve due to delivery hurdles to the meibomian glands. However, considering its consequences, it is important for ophthalmologists and optometrists to consider lid margin disease so that it will be identified and treated. Presently there is no cure for lid margin disease. Strategies are available to resolve acute flares and maintain disease control, but treatment is challenging because these interventions may be tedious, time-consuming, and must be continued long-term. Educating patients about the chronic nature of lid margin disease and its sequelae is important for increasing compliance that is the basis for effective management. Considering the prevalence of lid margin disease and its burden, there exists a need for an ocular therapeutic and therapeutic regimen for improving lid margin function and treating and preventing diseases related thereto. The present invention meets this need and other needs.

### SUMMARY OF THE INVENTION

The present invention provides topical ophthalmic formulations suitable for improving lid margin function which comprise a combination of ingredients capable of acting synergistically to normalize meibomian gland secretion (i.e., decrease meibomian secretion viscosity, increase secretions transparency and decrease the time between gland secretions) and relieve ocular discomfort, thereby treating and/or preventing at least one sign or symptom of lid margin disease. In particular, the formulations described herein comprise a broad spectrum antibiotic having both anti-infective and anti-inflammatory properties in a mucoadhesive vehicle suitable for intermittent and/or repeated long term use for the treatment and/or prevention of lid margin disease and diseases associated therewith (e.g., dry eye disease, chronic conjunctitivits, chalazia and keratitis).

Further provided are methods of treating lid margin disease (e.g., anterior and posterior blepharitis, meibomianitis, meibomian gland dysfunction) and diseases associated therewith (e.g., dry eye disease), comprising administering to the eyelid margin, to the front of the eye, under the upper eyelid, on the lower eyelid, to the lacrimal gland or the cul-de-sac of said subject an effective amount of a topical ophthalmic formulation of the invention. In one embodiment, the ophthalmic formulation is administered at least once a day (e.g., once or twice a day or more) for up to at least three months (i.e., up to three months or longer). In another embodiment, the medication may be discontinued after three months of therapy and then re-instituted as needed for prolonged periods of time. In yet another embodiment the medication may be used continuously, as determined by the patient's clinical response. Optionally, the ophthalmic formulation is gently massaged into the eyelid of the subject following topical administration to the eyelid margin, to the front of the eye, under the upper eyelid, on the lower eyelid, to the lacrimal gland or the cul-de-sac. In some embodiments, the topical ophthalmic formulation of the invention is administered in conjunction with a palliative therapeutic, such as lid hyperthermia, eyelid hygiene, and/or nutritional supplements (e.g., omega-3, fish oil, flaxseed oil).

In any of the methods or formulations, the broad-spectrum anti-infective agent includes but is not limited to a tetracycline family antibiotic (e.g., tetracycline, doxycycline, minocycline or any derivative thereof). In a particular embodiment, the anti-infective agent is doxycycline or a derivative thereof. In another particular embodiment, the anti-infective agent is tetracycline or minocycline, or any derivatives thereof. Examples of mucoadhesive vehicles suitable for use in the methods or formulations of the invention include but are not limited to aqueous polymeric suspensions comprising one or more polymeric suspending agents including without limitation dextrans, polyethylene glycol, polyvinylpyrolidone, polysaccharide gels, Gelrite®, cellulosic polymers, and carboxy-containing polymer systems. In one preferred embodiment, the polymeric suspending agent comprises a crosslinked carboxy-containing polymer (e.g., polycarbophil). Examples of cross-linked carboxy-containing polymer systems suitable for use in the topical ophthalmic formulations of the invention include but are not limited to Noveon AA-1, Carbopol®, and/or DuraSite®.

Also provided by the present invention are methods for delivery a therapeutic agent to the meibomian gland orifice of a subject comprising: (a) formulating the therapeutic agent in a mucoadhesive vehicle comprising a polymeric suspending agent, including without limitation dextrans, polyethylene glycol, polyvinylpyrolidone, polysaccharide gel, Gelrite®, cellulosic polymers, carboxy-containing polymer systems, and any combination thereof; (b) administering the formulation of step (a) to the eyelid margin, to the front of the eye, under the upper eyelid, on the lower eyelid, to the lacrimal gland, or the cul-de-sac of said subject; and (c) massaging the formulation of step (a) into the eyelid of said subject after the application step (b). The non-aqueous component provides acute protection of the meibomian gland orifice, optimizes efficacy of supporting the tear film of the ocular surface, and increases the dwell time of the active agent to the meibomian gland orifice thereby increasing therapeutic efficacy of the active agent, while the administration to the eyelid margin, to the front of the eye, under the upper eyelid, on the lower eyelid, to the lacrimal gland or the cul-de-sac of said subject and the massaging of the formulation into the eyelid increases the delivery of the active agent to the meibomian gland orifice.

Other features and advantages of the invention will become apparent from the following detailed description and claims.

### DETAILED DESCRIPTION

For convenience, before further description of the present invention, certain terms employed in the specification, examples, and appended claims are collected here. These definitions should be read in light of the remainder of the disclosure and understood as by a person of skill in the art.

The term “abnormal meibomian gland secretion” refers to a meibomian gland secretion with increased viscosity, decreased secretion, higher melting temperature, increased inflammatory composition, inspissation of the meibomian glands, opacity, color and/or an increased time (refractory period) between gland secretions.

The term “blepharitis” refers to a disorder comprising inflammation of the lid margin associated with abnormal meibomian gland secretions (known as meibum) in which meibomian gland secretions accumulate and obstruct the meibomian gland duct, causing inflammation of the gland and bacterial colonization. Lid keratinization, lid margin rounding, obscuration of the grey line, increased lid margin transparency, and increased vascularity are often observed. Unless otherwise indicated, the term blepharitis, as used herein, includes anterior and posterior blepharitis, and bacterial, parasitic, seborrheic, eczematoid, and neoplastic forms of blepharitis.

The term “dry eye disease” as used herein refers to inadequate tear production, abnormal tear composition and/or abnormalities of the lipid tear layer, encompassing the terms, but not limited to: qualitative dry eye, evaporative dry eye.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of lid margin irritation, or prevent or treat lid margin disease. The effective amount may vary depending on such factors as the disease or condition being treated, the particular composition being administered, or the severity of the disease or condition. One of skill in the art may empirically determine the effective amount of a particular agent without necessitating undue experimentation.

The term “mucoadhesive vehicle” refers to a polymer which provides prolonged residence time of dosage forms as well as enhanced drug bioavailability by mucoadhesion. “Mucoadhesion refers to interfacial force interactions between synthetic or natural polymeric materials serving as a dosage form and a mucus layer that covers a mucosal tissue.

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid (aqueous or non-aqueous) or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, such as abnormal meibomian gland secretions, is art-recognized, and refers to administration of a composition which reduces the frequency of, or delays the onset of, signs and/or symptoms of a medical condition in a subject relative to a subject which does not receive the composition.

The term “tetracycline family antibiotics” refers to a subclass of polyketides having an octahydrotetracene-2-carboxamide skeleton, and includes but is not limited to tetracycline, chlortetracycline, oxytetracycline, demeclocycline, doxycycline, lymecycline, meclocycline, methacycline, minocycline, and olitetracycline.

The term “treating” is an art-recognized term which refers to curing as well as ameliorating at least one symptom of any condition or disease.

### Lid Margin Disease

Lid margin disease can be defined as any abnormality of the lids and associated adnexal structures, including the accessory glands of the eyelid margin, the mucocutaneous junction, and the meibomian glands (Foulks). Blepharitis is a general term often used interchangeably with lid margin disease. It describes a spectrum of conditions characterized by inflammation of the eyelids and includes dermatologic diseases, allergic reactions, and infections.

Several different classification schemes for blepharitis exist, but a method that divides it into anterior blepharitis and posterior blepharitis based on the anatomic site of pathologic involvement is simple and clinically useful (Wilhemus). Accounting for the vast majority of cases of blepharitis, this categorization also reflects differences in etiology and characteristic clinical features.

Anterior blepharitis is defined as disease affecting predominantly the lid region at the base of the eyelashes. It encompasses both staphylococcal blepharitis and seborrheic blepharitis, although a mixed presentation with both types of blepharitis is common. Posterior blepharitis involves the lid margin posterior to the eyelashes. It usually results from disorders of the meibomian glands and is also referred to as meibomian gland dysfunction (MGD) or meibomianitis. The meibomian glands are enlarged sebaceous glands that produce meibum, a lipid secretion that is an important component of the normal tear film. Posterior blepharitis may involve either obstruction of the meibomian glands with stagnation of meibomian gland secretions and inspissation of the duct orifices (meibomianitis) or overproduction of meibomian gland secretions (meibomian seborrhea).

Bacterial colonization of the eyelids is present in both patients with blepharitis as well as individuals with healthy eyelids. Coagulase negative staphylococci, Corynebacterium spp, Staphylococcus epidermidis, and Propionibacterium acnes comprise the most frequently isolated microflora (Driver). Colonization by Staphylococcus aureus is less prevalent, although this organism appears to be more common in patients with staphylococcal blepharitis than in normal individuals (McCulley). Both S. aureus and S. epidermidis are thought to play an etiologic role in staphylococcal blepharitis. The exact mechanisms are yet to be fully defined, but may involve direct infection, reactions to bacterial exotoxins, or an antigen-related immunologic reaction (AAO, Smith).

Epithelial hyperkeratinization leading to narrowing of the meibomian gland duct is considered important in the pathogenesis of posterior blepharitis. There is also evidence that the biochemical composition of the meibomian secretions is altered in patients with posterior blepharitis compared to normal controls. The altered lipid secretions stagnate within the gland where they may provide a medium for bacterial growth and promote tissue inflammation. The underlying cause for the qualitative changes in the meibomian secretions is unproven, but may be mediated through the action of bacterial lipases (Smith). In that regard, it has been reported that while the prevalence of S. epidermidis eyelid colonization is similar in patients with MGD and controls, there may be differences between these populations in strain type relating to differences in bacterial lipase activity (Dougherty).

There is significant overlap in the clinical features of anterior and posterior blepharitis as both conditions manifest with signs and symptoms relating to irritation and inflammation of the eyelids, conjunctiva, and cornea (Smith, AAO). However, certain findings, including associations with dermatologic disease, are more characteristic of the different types of blepharitis.

Staphylococcal blepharitis presents with scaling (collarettes), eyelid crusting, and debris formation along the base of the lashes. Affected patients may complain of burning, itching, light sensitivity, and foreign body sensation. These symptoms and eyelid crusting are usually worst in the morning. Other clinical signs include lid margin thickening and hyperemia. Chronic inflammation from long-standing disease can lead to eyelid scarring and lash changes, including lash loss, breakage, and/or misdirection. Ulceration affecting the base of the lash follicles is rare but can develop during a severe, acute exacerbation. Corneal complications can occur and include inferior or diffuse punctate epithelial erosions, marginal infiltrates, scarring, neovascularization, pannus, thinning and perforation, phlyctenules, and Salzmann's nodules. Conjunctival findings include mild to moderate hyperemia associated with chronic papillary conjunctivitis. Other sequelae associated with staphylococcal blepharitis include pustules, recurrent chalazia, external and internal hordeola, and tear film instability. Patients with staphylococcal blepharitis generally do not have associated dermatologic disease, although atopy may be present (AAO).

Seborrheic blepharitis is also manifested by lid erythema, scaling, and lash abnormalities, although the scales have a greasy appearance, the lashes tend to be stuck together, and the anterior lid margin may have a shiny appearance. Inflammation is less severe in seborrheic blepharitis compared with staphylococcal blepharitis, and patients may be more likely to present with a longer duration of symptoms without exacerbations. Conjunctivitis or keratitis have been reported in a minority of patients with seborrheic blepharitis (AAO). When present, the keratitis generally involves the inferior third of the cornea and is manifested by punctate epithelial erosions. Seborrheic dermatitis is a common comorbidity.

Patients with posterior blepharitis also commonly experience chronic pain and complain of burning, itching, tearing, and foreign body that is worse upon awakening. These patients also complain of decreased or fluctuation in vision. Eyelid examination reveals inflammation of the orifices of the meibomiam glands and thickening of the posterior lid margin. The meibomian gland secretions have a thickened, toothpaste-like appearance and may be only poorly expressed when digital pressure is applied to the lid margin. Over time, the meibomian glands may atrophy and eyelid scarring can develop. As with staphylococcal blepharitis, chalazia is common and patients may also report mattering/crusting of the lids and lashes. Patients with posterior blepharitis have an increased risk of external eye infections, particularly recurrent bacterial conjunctivitis. In fact, most bacterial conjunctivitis is blepharoconjunctivitis with the infection starting in the lid and then spreading to the tear film and conjunctiva. In these patients, treatment of the precipitating lid margin disease will resolve the problem definitively, while treating the conjunctival infection only with a topical antibiotic may afford only temporary improvement followed by recurrent infection.

Clinical features of meibomian seborrhea include the presence of oil globules overlying the orifices of the meibomian glands and an oily, foamy tear film. This form of MGD may be associated with seborrheic dermatitis and rosacea (AAO, Driver). Blepharitis and Dry Eye Disease

Dry eye disease has been reported as present in 56% of patients with blepharitis, 48% of patients with obstructive MGD, and 79% of patients with seborrheic MGD (Mathers). The frequent association between posterior blepharitis and dry eye disease is not surprising considering that the lipids secreted by the meibomian glands are necessary for normal tear film stability and function. However, dry eye disease may arise through alternative mechanisms. It has been theorized that the altered meibomian gland secretions in patients with posterior blepharitis may have a direct, irritant effect on the ocular surface tissues (Smith). The bacterial flora associated with blepharitis may also induce tear film abnormalities and ocular surface inflammation through multiple pathways, including release of exotoxins, induction of pro-inflammatory cytokines and other chemical mediators, and provocation of hypersensitivity reactions (Foulks).

Patients with posterior blepharitis tend to have an evaporative dry eye condition relating to a deficiency in tear quality rather than quantity. Insufficient production of the lipid components of the tear film in patients with MGD is associated with excessive water loss from the ocular surface in the presence of normal aqueous production by the lacrimal glands. However, keratoconjunctivitis sicca has been reported in up to 40% of patients with MGD, in half of patients with staphylococcal blepharitis, and in about one-third of patients with seborrheic blepharitis (AAO. Smith, Bowman).

### Blepharitis in the Surgical Candidate

An important consideration in patients with blepharitis is its potential to impact the outcomes of ocular surgery. An already compromised tear film in patients with blepharitis may be further degraded by a variety of surgery-related factors, including severing of corneal nerves by surgical incisions or a LASIK flap, surgically-induced inflammation, and irritation caused by topically instilled medications. As a result, patients may be more debilitated by symptoms of dry eye and achieve a poorer functional result considering that an optimal precorneal tear film is requisite for quality vision. Patients with uncontrolled blepharitis undergoing LASIK are also at risk for trapping of meibomian debris beneath the LASIK flap. The FDA lists blepharitis as a risk factor that patients should be screened for by their surgeons prior to LASIK eye surgery. In a study analyzing findings in patients presenting to a tertiary care center because of dissatisfaction postLASIK, dry eye/blepharitis ranked as the most common diagnosis (Levison).

The risk of postoperative infections may also be increased in patients with blepharitis considering that bacterial flora in the lids and lashes are a primary source of pathogens in these infections. Univariate analysis in a case-control study found that blepharitis was associated with late-onset infection after glaucoma filtering surgery (Jampel). The authors noted the increased bacterial load may predispose to infection.

### Diagnosis of Blepharitis

Diagnosis is the first step in the effective management of lid margin disease. Blepharitis may be the most common ophthalmologic disease. It is a frequent underlying cause of other common disorders, including dry eye disease and recurrent conjunctivitis, as well as an important risk factor for suboptimal outcomes after ocular surgery. Therefore, ophthalmologists must remember that all patients deserve a thorough lid and ocular surface examination to identify chronic blepharitis and its sequelae.

The diagnosis of blepharitis is often overlooked because its symptoms tend to be nonspecific or interpreted as related to dry eye disease. Certainly, dry eye disease is a common comorbidity in patients with blepharitis, and it is appropriate that the office evaluation include assessment of the tear film and ocular surface. However, blepharitis may be the underlying cause of any identified abnormalities. Other entities to consider in the differential diagnosis of patients who present with eyelid inflammation include entropion, ectropion, floppy eyelid syndrome, lid imbrication syndrome, mucous fishing syndrome, previous lid surgery or trauma, and allergic eye disease. Acute infections of bacterial, viral, or parasitic origin may present as blepharitis, albeit rarely. Various benign and malignant neoplasms may also masquerade as blepharitis. The diagnosis of a tumor may be suspected when the lid disease involves only one eye rather than being bilateral and also if the condition persists after conventional treatment. Early diagnosis is important because of the serious and even life-threatening nature of these neoplasms. Eyelid inflammation may also be medication-induced, with oral isotretinoin being one of the most important culprits (Fraunfelder).

Diagnosis of blepharitis is often made by a combination of patient history, clinical evaluation and ocular surface examination. Risk factors for blepharitis that are often assessed when taking a patient's history include but are not limited to the duration and nature of patient's symptoms, exacerbation by any environmental or other extrinsic factors (eg., low humidity, smoke, allergens, contact lens wear), a history of ocular surgery or trauma, exposure to scabies or other infections that may affect the external eye, medication use, and presence of dermatologic diseases associated with blepharitis (rosacea, atopy, seborrhea). Clinical evaluation for blepharitis includes but is not limited to gross examination of the lid margin for inflammation and evidence of foreign matter or debris (e.g., using a slit-lamp to examine the lid margins s for pustules, lash loss, or other abnormalities); eversion of the lids for examination of the meibomian gland orifices and the nature of the secretions. Patients with primary meibomitis exhibit discolored meibomian secretions, inspissation and inflammation near the gland orifices, and ductile dilation. Telangiectasia, hyperemia, and thickening can be seen as signs of chronic inflammation of the lid margin, and there may be secondary anterior lid margin involvement.

Upon examination of the ocular surface, identification of a soapsuds-like material sitting on the tear film in the inferior lid margin is a key finding as it is pathognomic for MGD. This foamy material arises as a result of bacterial lipases cleaving the meibomian gland secretions into fatty acids and mono- and diglycerides. Presence of conjunctival and corneal involvement should also be investigated. Conjunctival findings include injection, papillary hypertrophy, and staining. A supravital dye, either lissamine green or rose bengal, is more useful than fluorescein for the purpose of identifying conjunctival damage. The features of the conjunctival staining in patients with blepharitis overlap with those associated with aqueous deficiency dry eye disease. However, the corneal staining in patients with anterior or posterior blepharitis is often localized to the inferior region where the staphylococcus fall into the tear film and are absorbed into the tear lake.

The diagnostic workup for any patient who complains of foreign body sensation and ocular irritation should also include a Schirmers test to diagnose aqueous deficiency dry eye. An abnormal Schirmers test result does not rule out comorbid MGD, but the finding of normal aqueous production should prompt further evaluation for blepharitis as an underlying etiology for the symptomatic complaints. Since patients with dry eye disease associated with MGD have poor quality tears, they also tend to have a shortened fluorescein tear breakup time even in the presence of normal aqueous production (Pflugfelder).

Complaints of waxy, filmy vision that is worse on awakening in the morning is one of the most important findings for differentiating between blepharitis and aqueous deficiency dry eye disease as the latter patients are generally least symptomatic in the morning and experience worsening over the course of the day as the tear film dehydrates. Failure to respond to traditional conservative therapy for dry eye disease, including artificial tears and topical cyclosporine ophthalmic emulsion, also suggests the need for a more careful evaluation to identify blepharitis as the underlying diagnosis.

Biopsy to diagnose malignancy and cultures for microbiologic testing may be considered on a case-by-case basis depending on the individual features of the patient, including response to treatment.

### Management of Blepharitis

Blepharitis is a chronic, incurable disorder requiring ongoing care where the goal is to achieve disease control in order to alleviate associated signs and symptoms, improve quality of life, and prevent complications that can result in permanent tissue damage and even vision loss. Addressing blepharitis preoperatively is also important to reduce complications and optimize the functional outcome for their surgical patients.

Current treatment options for blepharitis typically incorporate several different palliative and therapeutic measures, including lid hyperthermia, lid hygiene, anti-inflammatory agents, nutritional supplements, and topical and/or oral antibiotics, that aim to clear the lid margin from debris and infection as well as reduce inflammation.

Lid hyperthermia, involving application of warm compresses to the eyelids, is a mainstay of therapy for both anterior and posterior blepharitis. In anterior blepharitis, the heat acts to loosen adherent plaques while in posterior blepharitis it induces melting of the meibomian gland secretions and helps to relieve blockage of the gland orifices. Patients with posterior blepharitis may be advised to gently massage the lids following lid hyperthermia to express secretions from the meibomian glands.

Eyelid hygiene is also a fundamental component of treatment for blepharitis. It is particularly important in patients with anterior blepharitis where it is helpful for removing deposits from the lid margin and reducing bacterial colonization of the eyelids. However, it also plays a role in patients with posterior blepharitis as it can clean the lids of free fatty acids and bacteria that have been implicated in the pathogenesis of the disorder and associated dry eye disease. Cleansing should be directed to the lid margin and base of the lashes, not just the eyelid skin, and is preferably performed after lid hyperthermia.

Anti-inflammatory treatment with topical corticosteroids has a limited role in the treatment of chronic blepharitis. It may be prescribed to gain faster control of more severe inflammation, such as in a patient with an active chalazia/hordeolum, marginal infiltrative keratitis, or conjunctival or corneal phlyctenules. However, risks of corticosteroid treatment include lid atrophy, glaucoma, cataract, and severe opportunistic infections. Therefore, selection of a lower potency corticosteroid, or other site-specific agent should be considered, and the duration of treatment should be limited (McCulley, Dougherty). Topical cyclosporine offers an alternative agent for treating acute inflammation. Studies evaluating cyclosporine in patients with posterior blepharitis found that it was associated with decreased meibomiam gland inclusions along with improvements in meibomian gland secretion quality, symptoms, fluorescein staining, and lid telangiectasia (Perry, Rubin).

Preliminary evidence indicates that oral supplementation with essential fatty acids (e.g., flaxseed) may thin and improve the quality of the meibomian gland secretions in patients with blepharitis (Lahners, Boerner). The rationale for this strategy relates to evidence that dietary intake of fatty acids affects the lipid composition of meibomian gland secretions (Sullivan).

Since bacteria, especially Staphylococcus spp, are considered to play a role in posterior as well as anterior blepharitis, antibiotics with antistaphylococcal activity may be appropriate treatment for any of these patients. Historically, ointment preparations, particularly bacitracin and erythromycin, have been most widely used for the treatment of acute anterior blepharitis. However, such antibiotic ointments are not generally effective for treating posterior blepharitis because the active ingredients in these petrolatum based-vehicles do not penetrate significantly into the eyelid margins and cause blurring of vision that is disturbing to patients. Antibiotics formulated in conventional ophthalmic drops penetrate into the conjunctiva and tear film, but bioavailability to the lid margin and penetration into the eyelid tissue is still poor.

Oral tetracycline family antibiotics have been prescribed for patients with posterior blepharitis when their disease is not adequately controlled by eyelid hygiene and lid hyperthermia, and it may be particularly considered for patients with co-morbid rosacea. In addition to having antimicrobial effects, tetracyclines provide anti-inflammatory activity (Perry) separate and distinct from their antibiotic effects and have been reported to reduce bacterial lipase and collagenase production. (Frucht-Perry). Unfortunately, the tissue levels of antibiotics achieved with oral dosing are much lower than those that can be attained with local, topical application. Oral tetracycline treatment is also accompanied by the potential for systemic side effects, including sun sensitivity, vaginal yeast infections, GI disturbances, and possibly the risk of breast cancer. In addition, these agents are contraindicated for use in women who are pregnant or nursing, and may interact with oral contraceptives and warfarin. They should also not be used in young children because of risks to bone and teeth.

### Pharmaceutical Compositions

The invention features topical pharmaceutical compositions comprising an anti-infective/anti-inflammatory agent (e.g., a tetracycline family antibioitic) and a mucoadhesive vehicle useful for treating lid margin disease. An effective amount of the formulations of the invention may be used to improve lid margin function, thereby treating diseases associated therewith (e.g., blepharitis, meibomianitis, meibomian gland dysfunction, and dry eye). Signs and symptoms of abnormal lid margin function include but are not limited to increased meibomian secretion viscosity, decreased secretion, higher melting temperature, increased inflammatory composition, inspissation of the meibomian glands, opacity, color, as well as an increase in the time (refractory period) between gland secretions Signs and symptoms of diseases associated with lid margin disease include but are not limited to dry eye, burning, fluctuating vision, redness of the eyes, itching and/or irritation of the eyelid margins and edema, foreign body sensation, and matting of the lashes.

Preferably, the anti-infective agent has both broad spectrum anti-infective activity and anti-inflammatory properties, as well as high tissue penetration. The efficacy of the formulations described herein is attributed, in part, to the synergistic effect of the combination of ingredients in them. The anti-infective/anti-inflammatory agent relieves, inhibits, prevents, or otherwise decreases the signs and symptoms associated with meibomian gland disease by improving lid margin function. The mucoadhesive vehicle provides increased contact time and adhesion of the anti-infective agent to the lid margin, and thereby increased penetration of the anti-infective agent into the eyelid tissue and meibomian glands. As such, the pharmaceutical compositions of the invention has enhanced therapeutic efficacy and duration of action over other formulations of anti-infective agents that are not combined with such mucoadhesive vehicles as described herein and do not have an anti-inflammatory effect. The enhanced therapeutic efficacy of the compositions of the invention allow for less frequent dosing than anti-infective agents combined with conventional vehicles while achieving therapeutic anti-microbial and anti-inflammatory levels.

Exemplary anti-infective agents include, but are not limited to tetracycline family antibiotics (e.g., tetracycline, doxycycline, minocycline) or derivatives or analogs thereof, aminoglycoside antibiotics, bacitracin, erythromycin, neomycin, trimethoprim, sulfa drugs, fluoroquinolones, and chlorophenicol. In a preferred embodiment, the anti-infective agent is doxycycline. In another preferred embodiment, the anti-infective agent is a tetracycline family antibiotic (e.g., tetracycline, minocycline, doxycycline, or any combination thereof or derivative thereof).

Pharmaceutical ophthalmic formulations typically contain an effective amount, e.g., about 0.1% to about 1% w/v, preferably about 0.15% to about 0.85% w/v, more preferably about 0.25% to about 0.75% w/v, even more preferably about 0.3% to about 6% w/v of an anti-infective agent suitable treating and/or preventing meibomian gland disease. The concentration within the ocular tissue is desired to be at least 0.25 μg/g which is the MIC for many antibiotics, however higher concentrations are preferable (e.g., at least 1 μg/g, more preferably at least 10 μg/g) to eradicate resistant organisms and to provided more rapid resolution of an infectious process. In addition, the higher the concentration of antibiotic which has a concentration dependent anti-inflammatory effect such as a tetracycline antibiotic results in greater anti-inflammatory effect. The amount of anti-infective agent actually supplied to the external eye surface will almost always be much higher than the tissue concentration. This reflects the penetration hold up of the anti-infective agent by the outer tissue layers of the eye and that penetration is to some extent concentration driven. Thus, supplying greater amounts to the exterior will drive more anti-infective agent into the tissues.

The pharmaceutical compositions of the invention described above may additionally comprise or be administered in conjunction with (simultaneously or sequentially) one or more additional active ingredients, including, but not limited to, an anti-infective agents (e.g., antibiotics, antivirals, antifungals), anti-inflammatory agents (including steroidal and non-steroidal anti-inflammatories), vasoconstrictors, antiallergenic agents, anesthetics, analgesics, and dry eye agents (e.g. secretagogues, mucomimetics, polymers, lipids, antioxidants).

The pharmaceutical compositions of the invention may be formulated as an aqueous suspension or solution. However, other dosage forms for topical administration to the external eye surface may be used (e.g., ointments, suspensions, viscous or semi-viscous gels, or other types of solid or semi-solid compositions).

Any of a variety of pharmaceutically acceptable carriers or vehicles that are compatible with the eye may be used in the formulations of the present invention. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils such as castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

Preferably the carrier is capable of increasing the therapeutic efficacy of the anti-infective agent by extending the residence time of the active agent to the meibomian gland orifice. In a particular embodiment, the carrier is a mucoadhesive. In particular embodiment, the compositions of the invention are formulated as an aqueous polymeric suspension. Typically the anti-infective agent is in suspension although it is possible for the anti-infective agent to be in solution (water soluble) or both in solution and in suspension. The polymeric suspending agent is preferably a suspension (i.e. water insoluble and/or water swellable), although water soluble suspending agents may also be used. The suspending agent serves to provide stability to the suspension and to increase the residence time of the dosage form on the eye. It can also enhance the sustained release of the anti-infective agent in terms of both longer release times and a more uniform release curve.

Examples of polymeric suspending agents include dextrans, polyethylene glycols, polyvinylpyrolidone, polysaccharide gels, Gelrite®, cellulosic polymers like hydroxypropyl methylcellulose, and carboxy-containing polymers such as polymers or copolymers of acrylic acid, as well as other polymeric demulcents. A preferred polymeric suspending agent is a water swellable, water insoluble polymer, especially a crosslinked carboxy-containing polymer.

Crosslinked carboxy-containing polymers used in practicing this invention are, in general, well known in the art. In a preferred embodiment such polymers may be prepared from at least about 90% and preferably from about 95% to about 99.9% by weight, based on the total weight of monomers present, of one or more carboxy-containing monoethylenically unsaturated monomers (also occasionally referred to herein as carboxy-vinyl polymers). Acrylic acid is the preferred carboxy-containing monoethylenically unsaturated monomer, but other unsaturated, polymerizable carboxy-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

Such polymers may be crosslinked by a polyfunctional crosslinking agent, preferably a difunctional crosslinking agent. The amount of crosslinking should be sufficient to form insoluble polymer particles, but not so great as to unduly interfere with sustained release of the anti-infective agent. Typically the polymers are only lightly crosslinked. Preferably the crosslinking agent is contained in an amount of from about 0.01% to about 5%, preferably from about 0.1% to about 5.0%, and more preferably from about 0.2% to about 1%, based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, preferably alkenyl ether groupings containing terminal H2C≡C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, the entire contents of which are incorporated herein by reference. Diolefinic non-hydrophilic macromeric crosslinking agents having molecular weights of from about 400 to about 8,000, such as insoluble di-acrylates and polyacrylates and methacrylates of diols and polyols, diisocyanate-hydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, the entire contents of each Patent being incorporated herein by reference.

The crosslinked carboxy-vinyl polymers may be made from a carboxy-vinyl monomer or monomers as the sole monoethylenically unsaturated monomer present, together with a crosslinking agent or agents. Preferably the polymers are ones in which up to about 40%, and preferably from about 0% to about 20% by weight, of the carboxy-containing monoethylenically unsaturated monomer or monomers has been replaced by one or more non-carboxyl-containing monoethylenically unsaturated monomer or monomers containing only physiologically and ophthalmically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat No. 4,548,990, the entire contents of which are incorporated herein by reference, for a more extensive listing of such additional monoethylenically unsaturated monomers.

Particularly preferred polymers are lightly crosslinked acrylic acid polymers wherein the crosslinking monomer is 2,3-dihydroxyhexa-1,5-diene or 2,3-dimethylhexa-1,5-diene. Preferred commercially available polymers include polycarbophil (Noveon AA-1) and Carbopol®. Most preferably, a carboxy-containing polymer system known by the tradename DuraSite®, containing polycarbophil, which is a sustained release topical ophthalmic delivery system that releases the drug at a controlled rate, is used in the aqueous polymeric suspension composition of the present invention.

The crosslinked carboxy-vinyl polymers used in practicing this invention are preferably prepared by suspension or emulsion polymerizing the monomers, using conventional free radical polymerization catalysts, to a dry particle size of not more than about 50 μm in equivalent pherical diameter; e.g., to provide dry polymer particles ranging in size from about 1 to about 30 gm, and preferably from about 3 to about 20 gm, in equivalent spherical diameter. Using polymer particles that were obtained by mechanically milling larger polymer particles to this size is preferably avoided. In general, such polymers will have a molecular weight which has been variously reported as being from about 250,000 to about 4,000,000, and from 3,000,000,000 to 4,000,000,000.

In the most preferred embodiment of the invention, the particles of crosslinked carboxy-vinyl polymer are monodisperse, meaning that they have a particle size distribution such that at least 80% of the particles fall within a 10 μm band of major particle size distribution. More preferably, at least 90% and most preferably at least 95%, of the particles fall within a 10 μm band of major particle size distribution. Also, a monodisperse particle size means that there is no more than 20%, preferably no more than 10%, and most preferably no more than 5% particles of a size below 1 μm. The use of a monodispersion of particles will give maximum viscosity and an increased eye residence time of the ophthalmic medicament delivery system for a given particle size. Monodisperse particles having a particle size of 30 μm and below are most preferred. Good particle packing is aided by a narrow particle size distribution.

The aqueous polymeric suspension normally contains 0.05 to 1%, preferably 0.1 to 0.5%, more preferably 0.1 to 0.5%, of the anti-infective agent and 0.1 to 10%, preferably 0.5 to 6.5% of a polymeric suspending agent. In the case of the above described water insoluble, water-swellable crosslinked carboxy-vinyl polymer, a more preferred amount of the polymeric suspending agent is an amount ranging from 0.5 to 2.0%, preferably from 0.5% to about 1.2%, and in certain embodiments from 0.6 to 0.9%, based on the weight of the composition. Although referred to in the singular, it should be understood that one or more species of polymeric suspending agent such as the crosslinked carboxy-containing polymer can be used with the total amount falling within the stated ranges. In one preferred embodiment, the composition contains 0.6 to 0.8% of a polycarbophil such as NOVEON AA-1.

In one embodiment, the amount of insoluble lightly crosslinked carboxy-vinyl polymer particles, the pH, and the osmotic pressure can be correlated with each other and with the degree of crosslinking to give a composition having a viscosity in the range of from about 500 to about 100,000 centipoise, and preferably from about 1,000 to about 30,000 or about 1,000 to about 10,000 centipoise, as measured at room temperature (about 25° C.) using a Brookfield Digital LVT Viscometer equipped with a number 25 spindle and a 13R small sample adapter at 12 rpm. Alternatively, when the viscosity is within the range of 500 to 3000 centipoise, it may be determined by a Brookfield Model DV-11+, choosing a number cp-52 spindle at 6 rpm.

When water soluble polymers are used as the suspending agent, such as hydroxypropyl methylcellulose, the viscosity will typically be about 10 to about 400 centipoise, more typically about 10 to about 200 centipoises or about 10 to about 25 centipoise.

Aqueous polymeric suspensions of the present invention may be formulated so that they retain the same or substantially the same viscosity in the eye that they had prior to administration to the eye. Alternatively, they may be formulated so that there is increased gelation upon contact with tear fluid. For instance, when a formulation containing DuraSite® or other similar polyacrylic acid-type polymer is administered to the eye at a pH of less than about 6.7, the polymer will swell upon contact with tear fluid since it has a higher pH (around 7). This gelation or increase in gelation leads to entrapment of the suspended anti-infective agent, thereby extending the residence time of the composition in the eye. The anti-infective agent is released slowly as the suspended particles dissolve over time. All these events eventually lead to increased patient comfort and increased contact time of the anti-infective agent with the eye tissues, thereby increasing the extent of drug absorption and duration of action of the formulation in the eye.

The viscous gels that result from fluid eye drops typically have residence times in the eye ranging from about 2 to about 12 hours, e.g., from about 3 to about 6 hours. The agents contained in these drug delivery systems will be released from the gels at rates that depend on such factors as the drug itself and its physical form, the extent of drug loading and the pH of the system, as well as on any drug delivery adjuvants, such as ion exchange resins compatible with the ocular surface, which may also be present.

The compositions used to topically deliver the anti-infective agent of the present invention can be prepared from known or readily available materials through the application of known techniques by workers of ordinary skill in the art without undue experimentation. For example, where the anti-infective agent is a tetracycline family antibiotic, it may be commercially available or readily obtained by a worker skilled in the art through known reactions techniques.

The anti-infective-containing composition is topically applied to an eye of a human or non-human animal, the latter including cows, sheep, horses, pigs, goats, rabbits, dogs, cats, and other mammals. The composition can be applied as a liquid drop, ointment, a viscous solution or gel, a ribbon or as a solid. The composition can be topically applied, without limitation, to the eyelid margin, to the front of the eye, under the upper eyelid, on the lower eyelid, to the lacrimal gland and/or in the cul-de-sac. Preferably, the composition is massaged into the eyelid immediately after topical application. The application can be as a treatment of lid margin disease and diseases associated therewith (e.g., dry eye disesase) or as a preventive such as prior to surgery.

The active agents of the pharmaceutical compositions may be in the form of a pharmaceutically acceptable salt. For example, where the preferred anti-infective agent is poorly soluble in water, solubility is improved if converted to a salt form. Ointments and solid dosage forms can also be used as delivery compositions as are well known in the art. The concentration of anti-infective agent present in the ophthalmic composition depends upon the dosage form, the release rate, the dosing regimen, and the location and type of infection. Generally speaking, the concentration is from about 0.01 to 2%, more typically 0.1 to 1%, for fluid compositions and 0.5 to 50% for solid dosage forms, however, the compositions are not limited thereto.

The pharmaceutical ophthalmic compositions of the present invention, including both ointments and suspensions, have a viscosity that is suited for the selected route of administration. A viscosity in the range of from about 1,000 to 30,000 centipoise is useful for a drop. About 30,000 to about 100,000 centipoise is an advantageous viscosity range for ophthalmic administration in ribbon form. The viscosity can be controlled in many ways known to the worker skilled in the art. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Additional ingredients that may be included in the formulations of the invention include tonicity enhancers, preservatives, solubilizers, non-toxic excipients, demulcents, sequestering agents, pH adjusting agents, and co-solvents.

The pH may be adjusted by adding any physiologically and ophthalmically acceptable pH adjusting acids, bases or buffers to within the range of about 5.0 to 8.5. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof.

Tonicity is adjusted if needed typically by tonicity enhancing agents. Such agents may, for example be of ionic and/or non-ionic type. The osmotic pressure (π) of the aqueous ophthalmic composition is generally from about 10 milliosmolar (mOsM) to about 400 mOsM, more preferably from 260 to 340 mOsM. If necessary, the osmotic pressure can be adjusted by using appropriate amounts of physiologically and ophthalmically acceptable salts or excipients. Sodium chloride is preferred to approximate physiologic fluid, and amounts of sodium chloride ranging from about 0.01% to about 1% by weight, and preferably from about 0.05% to about 0.45% by weight, based on the total weight of the composition, are typically used. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above-stated range. Similarly, a sugar such as mannitol, dextrose, sorbitol, glucose and the like can also be used to adjust osmolality.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenyhnercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

### Methods of Use

The invention features methods of improving lid margin function and treating lid margin disease and diseases associated therewith (e.g., dry eye disease) in a subject comprising use of the ophthalmic formulations described above. For example, a method of treating lid margin disease may comprise administering to the eyelid margin, to the front of the eye, under the upper eyelid, on the lower eyelid, to the lacrimal gland and/or the cul-de-sac of a subject in need thereof a formulation comprising an effective amount of an anti-effective agent (e.g., a tetracycline family antibiotic) in a mucoadhseive vehicle, and gently massaging the formulation into the eyelid immediately after topical application for 5-7 days or greater, up to and including a month's duration. After discontinuing the medication for one month this process may be repeated indefinitely as needed. Depending on the clinical response patients may be treated on and off (i.e., one month on, one month off) for several months or continuously for several months, before discontinuing the medication for a one month trial.

By treating lid margin disease and diseases associated therewith using the formulations described herein (i.e., a mucoadhesive broad spectrum antibiotic with strong tissue penetration) there is an improvement in tear function, and quality of vision. In certain embodiments, the compositions may improve lid margin function by normalizing meibomian gland function. (i.e., decreasing the meibomian secretion viscosity, reducing meibomian gland inflammation, stabilizing the tear film, increasing secretions transparency to a colorless state and decreasing the time (refractory period) between gland secretions). In certain embodiments, the compositions of the invention may treat dry eye disease by penetrating into the meibomian gland and improving meibomian gland function, thereby optimizing the efficacy of supporting the tear film of the ocular surface. In certain embodiments, the compositions of the invention may treat or prevent dry eye disease by penetrating into the lacrimal glands and improving aqueous tear quality and quantity.

The high tissue penetration and efficacy afforded by the formulations of the invention (i.e., a mucoadhesive broad spectrum antibiotic with strong tissue penetration) enables such formulations to be dosed less frequently than anti-infective agents combined with conventional vehicles while adhering to the lid margin and achieving therapeutic anti-microbial and anti-inflammatory levels in the lid margin. In some embodiments, the therapeutic dosing regimen is administered in conjunction with (simultaneously or sequentially) with one or more palliative and/or therapeutic measures including but not limited to lid hyperthermia, eyelid hygiene, nutritional supplements, another anti-infective agent, an anti-inflammatory agent, or any combination thereof. Such administration may reduce at least one symptom of lid margin disease in a subject and may operate by improving lid margin function in the subject.

For example, for the treatment of anterior blepharitis, an effective amount of the ophthalmic formulation of the invention (e.g., a tetracycline family antibiotic in a mucoadhesive vehicle (e.g., Durasite®)) is topically administered, as previously described, at least once a day (e.g., once (QD) or twice a day (BID) or more) for up to at least three months (i.e., up to three months or longer) in conjunction with (simultaneously or sequentially) lid hygiene, lid hyperthermia, nutritional supplements (e.g., omega-3, fish oil, flaxseed oil), or any combination thereof.

In certain embodiments, topical administration of the formulations of the invention is discontinued for a period of time while other treatments are continued (e.g., lid hyperthermia, eyelid hygiene, treatment and anti-infective or anti-inflammatory agent, nutritional supplements, or a combination thereof. For example, for the treatment of posterior blepharitis, an effective amount of the ophthalmic formulation of the invention (e.g., a tetracycline family antibiotic in a mucoadhesive vehicle (e.g., Durasite®)) is topically administered, as previously described, at least once a day (e.g., once (QD) or twice a day (BID) or more) for up to at least three months (i.e., up to three months or longer) in conjunction with (simultaneously or sequentially) a palliative regimen including lid hygiene, lid hyperthermia, nutritional supplements (e.g., omega-3, fish oil, flaxseed oil), or any combination thereof. If significant improvement is not seen after three months, topical administration of the formulation of the invention may be stopped or continued with an effective amount of an identical oral tetracycline family antibiotic, anti-inflammatory corticosteroids and/or cyclosporine. In other certain embodiments, topical administration of the formulations of the invention is reinstated every other month as needed.

Where a series of applications are used in the dosing regimen, it is possible that one or more of the earlier applications will not achieve an effective concentration in the ocular tissue, but that a later application in the regimen will achieve an effective concentration. This is contemplated as being within the scope of topically applying an anti-infective agent in an effective amount.

The effective amount of active agent to include in a given formulation, and the efficacy of a formulation for improving lid margin function may be assessed by one or more of the following: slit lamp evaluation, fluorescein staining, tear film breakup time, and evaluating meibomian gland secretions quality (by evaluating one or more of secretion viscosity, secretion color, gland alignment, vascularity pattern, vascularity redness, hyperkeratinization, posterior lid edge, lash, mucocutaneous junction, perigland redness, gland geometry and gland height).

The effective amount of active agent(s) in the formulation will depend on absorption, inactivation, and excretion rates of the drug as well as the delivery rate of the active agent(s) from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention may vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing

The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

All publications and patents mentioned herein are hereby incorporated by reference in their entirety as if each individual publication or patent was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

American Academy of Ophthalmology. Preferred Practice Pattern. Blepharitis. Available at: http://www.aao.org/education/guidelines/ppp/upload/Blepharitis-2.pdf. Accessed Feb. 29, 2008.

Anon. LASIK Eye Surgery. Available at: http://www.fda.fov/cdrh/lasik/when.htm. Accessed Feb. 29, 2008.

Behrens A, Doyle J J, Stern L, et al. Dysfunctional tear syndrome: a Delphi approach to treatment recommendations. Cornea 2006; 25:900-7.

Boerner C F. Dry eye successfully treated with oral flaxseed oil. Ocular Surgery News. 2000; 10:147-8.

Bowman R W, Dougherty J M, McCulley J P. Chronic blepharitis and dry eyes. Int Ophthalmol Clin 1987; 27:27-35.

Brown N A, Bron A J, Harding J J, et al. Nutritional supplements and the eye. Eye 1998; 12:127-33.

Dougherty J M, McCulley J P. Bacterial lipases and chronic blepharitis. Invest Ophthalmol Vis Sci 1986; 27:484-91.

Foulks G N. Blepharitis: Lid margin disease and the ocular surface. 39-48Ocular Surface Disease: Medical and Surgical Management. Eds; Holland E J, Mannis M J. Springer-Verlag 2002 New York

Fraunfelder F T, Meyer S M. Systemic reactions to ophthalmic drug preparations. Med Toxicol Adverse Drug Exp 1987; 2:287-93.

Frucht-Pery J, Sagi E, Hemo I, Ever-Hadani P. Efficacy of doxycycline and tetracycline in ocular rosacea. Am J Ophthalmol 1993; 116:88-92.

Jampel H D, Quigley H A, Kerrigan-Baumrind L A, et al. Risk factors for late-onset infection following glaucoma filtration surgery. Arch Ophthalmol 2001; 119:1001-8.

Lahners W, Palay D, Jones D Improvement in chronic meibomitis with essential fatty acid supplementation. Invest Ophthal Vis Sci 1999; 40:S541.

Lemp M A, Mahmood M A, Weiler H H. Association of rosacea and keratoconjunctivitis sicca. Arch Ophthalmol 1984; 102:556-7.

Levison B A, Rapuano C J, Cohen E J, JHammersmith K M, Ayres B D, Laibson P R. Referrals to the Wills Eye Institute Cornea Service after laser in situ keratomileusis: reasons for patient dissatisfaction. J Catract Refract Surg 2008; 34:32-9.

Mathers W D. Why the eye becomes dry. CLAO J 2000; 26:159-165

McCulley J P, Dougherty J M, Deneau D G. Classification of chronic blepharitis. Ophthalmology 1982; 89:1173-80.

Perry H D, Doshi-Carnevale S, Donnenfeld E D, et al. Efficacy of commercially available topical cyclosporine A 0.05% in the treatment of meibomian gland dysfunction (posterior blepharitis). Cornea 2006; 25:171-5.

Perry H D, Hodes L W, Seedor J A, Donnenfeld E D, McNamara T F, Golub L F. Effects of doxycycline hyclate on corneal epithelial wound healing in the rabbit alkali burn model. Cornea 12:379-382, 1993

Pflugfelder S C, Tseng S C, Sanabria O, et al. Evaluation of subjective assessments and objective diagnostic tests for diagnosing tear-film disorders known to cause ocular irritation. Cornea 1998; 17:38-56.

Rubin M, Rao S N, Efficacy of topical cyclosporine 0.05% in the treatment of posterior blepharitis. J Ocul Pharmacol Ther 2006; 22:47-53.

Smith R E, Flowers Jr C W. Chronic blepharitis: a review. CLAO J 1995; 21:200-207.

Sullivan R M. Correlations between nutrient intake and the polar lipid profiles of meibomian gland secretions in women with Sjogren's syndrome. Presented at the Third International Conference on the Lacrimal Gland, Tear Film and Dry Eye Syndromes: Basic Science and Clinical Relevance. Maui, Hawaii; Nov. 15-18, 2000.

Wilhemus K R. Inflammatory disorders of the eyelid margins and eyelashes. Ophthalmol Clin North America 1992; 5:187-94.

What is claimed is:

1. A method of treating lid margin disease comprising administering to a subject in
need thereof an effective amount of a topical ophthalmic composition comprising a combination of:
a) an anti-infective which comprises anti-inflammatory properties; and
b) a mucoadhesive vehicle;
wherein said composition is topically administered at least once a day for up to at least three months to the eyelid margin, front of the eye, under the upper eyelid, on the lower eyelid, to the lacrimal gland or the cul-de-sac of said subject.

2. The method of claim 1 wherein the anti-infective agent is a tetracycline family antibiotic.

3. The method of claim 2, wherein the tetracycline family antibiotic is doxycycline.

4. The method of claim 2, wherein the tetracycline family antibiotic is tetracycline or minocycline.

5. The method of claim 1, wherein the mucoadhesive vehicle comprises a polymeric suspending agent selected from the group consisting of a dextran, a polyethylene glycol, a polyvinylpyrolidone, a polysaccharide gel, Gelrite®, a cellulosic polymer, and a carboxy-containing polymer system.

6. The method of claim 5, wherein the carboxy-containing polymer system comprises a crosslinked carboxy-containing polymer.

7. The method of claim 6, wherein the crosslinked carboxy-containing polymer is polycarbophil.

8. The method of claim 6, wherein the carboxy-containing polymer system is selected from the group consisting of Noveon AA-1, Carbopol®, DuraSite®, or a combination thereof.

9. The method of claim 1 wherein the lid margin disease is blepharitis.

10. The method of claim 9, wherein the blepharitis is the anterior form of blepharitis.

11. The method of claim 9, wherein the blepharitis is the posterior form of blepharitis.

12. The method of claim 1, wherein the method further comprises massaging the ophthalmic formulation into the eyelid following topical administration to the eyelid margin, to the front of the eye, under the upper eyelid, on the lower eyelid, to the lacrimal gland or the cul-de-sac of said subject.

13. The method of claim 1, wherein the method further comprises administering to said subject a palliative therapeutic selected from the group consisting of lid hyperthermia, eyelid hygiene, nutritional supplements, or any combination thereof, in conjunction with said topical ophthalmic composition.

14. A method of treating dry eye disease, comprising administering to a subject in need thereof an effective amount of a topical ophthalmic composition comprising a combination of:
a) an anti-infective agent which comprises anti-inflammatory properties; and
b) a mucoadhesive vehicle,
wherein said composition is topically administered at least once a day for up to at least three months to the front of the eye, under the upper eyelid, on the lower eyelid, to the lacrimal gland or the cul-de-sac of said subject.

15. A method for delivering a therapeutic agent to the meibomian gland orifice of a subject comprising:
(a) formulating a therapeutic agent in a mucoadhesive vehicle comprising a polymeric suspending agent selected from the group consisting of a dextran, a polyethylene glycol, a polyvinylpyrolidone, a polysaccharide gel, Gelrite®, a cellulosic polymer, a carboxy-containing polymer system, and any combination thereof;
(b) administering the formulation of step (a) to the eyelid margin, to the front of the eye, under the upper eyelid, on the lower eyelid, to the lacrimal gland or the cul-de-sac of said subject; and
(c) massaging the formulation of step (a) into the eyelid of said subject after the application step (b).
